(19) United States
(12) Patent Application Publication
(10) Pub. No.: US 2026/0365906 A1
(43) Pub. Date: Nov. 19, 2026

(54) LIQUID WEIGHTING AGENTS FOR OIL-BASED FLUIDS

(71) Applicant: Albemarle Corporation, Charlotte, NC (US)

(72) Inventors: Yunqi LIU, Baton Rouge, LA (US); Joseph BAKER, Baton Rouge, LA (US); Huaxiang YANG, Sugar Land, TX (US); Garrett NIELSEN, Baton Route, LA (US); Donovan THOMPSON, Prairieville, LA (US); Gilles L. NUMKAM, Conroe, TX (US); Tse-Chong WU, Conroe, TX (US)

(73) Assignee: Albemarle Corporation, Charlotte, NC (US)

(21) Appl. No.: 19/132,271

(22) PCT Filed: Nov. 15, 2023
(86) PCT No.: PCT/US2023/079898

§ 371 Date: May 22, 2025

Related U.S. Application Data
(60) Provisional application No. 63/427,156, filed on Nov. 22, 2022.
(60) Provisional application No. 63/537,601, filed on Sep. 11, 2023.

(51) Int. Cl. C09K 8/035; C07C 17/02; C09K 8/34; E21B 41/00

(52) U.S. Cl. CPC C09K 8/035; C07C 17/02; C09K 8/34; E21B 41/00

(57) ABSTRACT

This invention provides compositions that can be used as liquid weighting agents.

### TECHNICAL FIELD

This invention relates to liquid weighting agents for oilfield applications.

### BACKGROUND

In some oilfield operations, the well fluids need to have a relatively high density, so one or more ingredients are added to the well fluid to increase the density of the well fluid. Brines are typically used to increase the density of aqueous well fluids, but some well fluids are oil-based. In oil-based fluids, solid weighting agents are often used to increase the well fluid density, but solid weighting agents often block pores and decrease production from the formation.

Liquid weighting agents are an alternative to solid weighting agents for oil-based well fluids, but may not be miscible with the oil-based fluid, may form emulsions with the oil-based well fluid, and/or cause solids formation. There is a need for weighting agents that do not form emulsions and/or cause precipitation.

### SUMMARY OF THE INVENTION

This invention provides liquid weighting agents that increase the density of oil-based well fluids, and can provide high density fluids. To achieve the increased density, the liquid weighting agent is made part of the oil-based well fluid. The liquid weighting agents of the invention can increase the densities of an oil-based well fluid to about 14.5 pounds per gallon (1.74 kg/L) or more. In addition to increasing the density of the fluid in which they are present, liquid weighting agents permit use of increased drill speeds. Another advantage of these liquid weighting agents is a greater amount of oil produced from the formation. Still another advantage of the liquid weighting agents of the present invention is that they do not significantly alter the fluid loss properties of formulations in which they are present. Yet another advantage provided by some of the liquid weighting agents of the invention is a minimal effect on the viscosity of the fluids to which they are introduced or combined; in some instances, liquid weighting agents of the invention reduce the viscosity of the fluids to which they are introduced or combined, so a change of equipment to larger pumps is not required. Another advantage provided by the liquid weighting agents of the invention is that they can be a component of optically clear fluids.

An embodiment of this invention is a liquid weighting agent containing two or more bromine atoms and having a bromine content of about 50 wt % or more, which liquid weighting agent is selected from
a) a bromine-containing terminal alkene having three or more carbon atoms in the molecule
b) a bromine-containing internal alkene having four or more carbon atoms in the molecule;
c) a tribromocumene mixture comprising 2,4,5-tribromocumene and one or more unsubstituted tribromobenzenes, wherein 2,4,5-tribromocumene is about 70 wt % or more relative to the total weight of the tribromocumene mixture, and the unsubstituted tribromobenzenes are about 5 wt % or more, relative to the total weight of the mixture; and
d) any two or more of the foregoing liquid weighting agents.

Another embodiment of this invention is an oil-based well fluid comprising a liquid weighting agent selected from the bromine-containing compounds and/or mixtures described above.

These and other embodiments and features of this invention will be still further apparent from the ensuing description and appended claims.

### FURTHER DETAILED DESCRIPTION OF THE INVENTION

As used throughout this document, the phrase “aromatic carbon atom” refers to a carbon atom that is part of an aromatic ring, and the phrase “alkenyl carbon atom” refers to a carbon atom that is part of the double bond.

Throughout this document, both “ppg” and “lb/gal” are abbreviations for pounds per gallon.

As used throughout this document, the term “tribromocumene” refers to mixtures that are comprised predominately of 2,4,5-tribromoisopropylbenzene, which can also be called 2-(2,4,5-tribromophenyl)propane.

Some of the liquid weighting agents of this invention are usable as oil-miscible weighting agents (OMWA). As used throughout this document, the term “miscible” means that emulsions and/or separate phases are not observed in a 50:50 weight mixture of one or more of the liquid weighting agents of this invention (total of 50 wt %) and an oil (50 wt %) after the mixture has been kept at 0 °C for one week. Similarly, as used throughout this document, the term “partially miscible” means that emulsions and/or separate phases are not observed in a 50:50 weight mixture of one or more of the liquid weighting agents of this invention (total of 50 wt %) and an oil (50 wt %) after the mixture has been kept at room temperature for one week.

The liquid weighting agents of the present invention are generally miscible with oil-based well fluids. In the practice of this invention, the liquid weighting agents are often in a mixture with an oil. Oils with which the liquid weighting agents are optionally mixed are oils which usually have a viscosity of about 7 cP or less, and a density typically about 0.7 g/mL to about 0.9 g/mL. The amount of liquid weighting agent in the oil can be about 0.1 wt % to 100 wt %; when more than one liquid weighting agent is used, the amount refers to the total amount of liquid weighting agents. When there is 100 wt % liquid weighting agent of the invention, no other substance is present with the liquid weighting agent. Preferred amounts of liquid weighting agent in the oil depend on the desired density, and there is typically about 0.1 wt % to 100 wt % liquid weighting agent in a mixture with an oil, more often about 0.1 to about 99.9 wt %, preferably about 2 wt % to about 99.9 wt % liquid weighting agent in a mixture with an oil. The mixture of oil and the liquid weighting agent(s) usually have a viscosity of about 10 cP or less, and a density typically about 0.7 g/mL to about 1.8 g/mL.

The liquid weighting agents of this invention share some overall characteristics. All of the liquid weighting agents of the invention are bromine-containing hydrocarbons having two or more bromine atoms in the molecule, and a bromine content of about 45 wt % or more, often about 50 wt % or more, preferably about 55 wt % or more, still more preferably about 60 wt % or more, relative to the total weight of the liquid weighting agent. The densities of the liquid weighting agents of the invention are generally about 1.20 g/mL or more.

In some embodiments, the liquid weighting agent is a bromine-containing terminal alkene. The bromine-containing terminal alkene has three or more carbon atoms, preferably three to about six carbon atoms in the molecule. Suitable terminal alkenes include 1-propene, 1-butene, 1-pentene, 1-hexene, 1-heptene, 1-octene, 1-nonene, and 1-decene; 2-propene is a preferred terminal alkene. Preferably, the two bromine atoms are bound to alkenyl carbon atoms. When there are more than two bromine atoms in the molecule, it is preferred that two or more bromine atoms are bound to alkenyl carbon atoms. Bromine-containing terminal alkenes of this invention typically have a bromine content of about 35 wt % or more, preferably about 40 wt % or more, more preferably about 50 wt % or more, still more preferably about 55 wt % or more, and often have densities of about 1.20 g/mL or more, preferably about 1.30 g/mL or more, more preferably about 1.40 g/mL or more, still more preferably about 1.50 g/mL or more. A preferred bromine-containing terminal alkene in the practice of this invention is 1,2-dibromo-1-hexene.

In some embodiments, the liquid weighting agent is a bromine-containing internal alkene. The bromine-containing internal alkene has four or more carbon atoms, preferably four to about eight carbon atoms in the molecule. Suitable internal alkene moieties include 2-butene, 2-pentene, 2-hexene, 3-hexene, 2,4-hexadiene, 2-octene 3-octene, and 4-octene; preferred internal alkene moieties include 2-butene and 2,4-hexadiene. Preferably, the two bromine atoms are bound to alkenyl carbon atoms. When there are more than two bromine atoms in the molecule, it is preferred that two or more bromine atoms are bound to alkenyl carbon atoms. Bromine-containing internal alkenes of this invention typically have a bromine content of about 35 wt % or more, preferably about 40 wt % or more, more preferably about 50 wt % or more, still more preferably about 55 wt % or more, even more preferably about 60 wt % or more. The bromine content is preferably in the range of about 35 wt % to about 85 wt %, preferably about 40 wt % to about 80 wt %. The bromine-containing internal alkenes often have densities of about 1.20 g/mL or more, preferably about 1.30 g/mL or more, more preferably about 1.40 g/mL or more, still more preferably about 1.50 g/mL or more. Preferred bromine-containing internal alkenes in the practice of this invention include 2,3-dibromo-2-butene and 2,3,4,5-tetrabromo-2,4-hexadiene.

In some embodiments, the liquid weighting agent is a tribromocumene mixture comprising 2,4,5-tribromocumene and one or more unsubstituted tribromobenzenes. The 2,4,5-ribromocumene is about 70 wt % or more, preferably about 70 wt % to about 75 wt %, relative to the total weight of the tribromocumene mixture. The unsubstituted tribromobenzenes are about 5 wt % or more, preferably about 5 wt % to about 10 wt %, relative to the total weight of the tribromocumene mixture. The unsubstituted tribromobenzenes preferably comprise 1,2,4-tribromobenzene in an amount of about 5 wt % or more, preferably about 6 wt % or more, more preferably about 5 wt % to about 10 wt %, relative to the total weight of the tribromocumene mixture. Another tribromobenzene that is preferably present in the tribromocumene mixture is 1,3,5-tribromobenzene, preferably in an amount of about 0.75 wt % or more, more preferably 0.75 wt % to about 1.5 wt %, relative to the total weight of the tribromocumene mixture. Often, brominated cumene species other than 2,4,5-tribromocumene are also present in the tribromocumene mixture, often in a total amount of about 5 wt % or more, preferably about 5 wt % to about 15 wt %, relative to the total weight of the relative to the total weight of the tribromocumene mixture. Typically, the other brominated cumene species are 2,3,5-tribromocumene, 3,4,5-tribromocumene, and 2,3,4,5-tetrabromocumene. A particularly preferred tribromocumene mixture comprises 2,4,5-tribromocumene in an amount of about 70 to about 73 wt %, 2,3,5-tribromocumene in an amount of about 3 wt % to about 5 wt %, 3,4,5-tribromocumene in an amount of about 1 wt % to about 2.5 wt %, 2,3,4,5-tetrabromocumene in an amount of about 7.5 wt % to about 9.5 wt %, 1,2,4,5-tetrabromobenzene in an amount of about 0.5 wt % to about 1.5 wt %, 1,2,4-tribromobenzene in an amount of about 6 wt % to about 8 wt %, and 1,3,5-tribromobenzene in an amount of about 1.25 wt % to about 2.5 wt %, relative to the total weight of the tribromocumene mixture. The bromine-containing mixtures comprising 2,4,5-tribromocumene often have densities of about 1.20 g/mL or more, preferably about 1.30 g/mL or more, more preferably about 1.40 g/mL or more, still more preferably about 1.50 g/mL or more.

The tribromocumene mixtures of the invention comprising 2,4,5-tribromocumene and one or more unsubstituted tribromobenzenes are preferably prepared in accordance with the synthesis process detailed herein. Such preferred tribromocumene mixtures are prepared by a process comprising contacting cumene (isopropylbenzene) and a brominating agent, preferably elemental bromine, in the presence of an aluminum chloride catalyst. Preferably, about 2.5 moles to about 3.5 moles of the brominating agent are used per mole of cumene, and the aluminum chloride is used in a catalytic amount (typically about 0.2 wt % to about 0.6 wt %, preferably about 0.3 wt % to about 0.5 wt %, relative to the cumene). The process is normally conducted under an inert atmosphere (e.g., nitrogen, argon, or helium) in a liquid organic medium, typically a solvent or mixture of solvents in which cumene is soluble. Non-limiting examples of suitable solvents for this purpose include halogenated organic solvents, including without limitation bromochloromethane, dibromomethane, mixtures of the foregoing, and the like. In another aspect of the invention, other organic solvents such as, e.g., cyclohexane, cycloheptane, nitromethane and the like are additional suitable organic solvents.

Combinations of any two or more of the above liquid weighting agents can be used in the practice of this invention.

Some of the liquid weighting agents of the invention have enough high-temperature thermal stability that they can be used in HPHT (high pressure, high temperature) conditions. HPHT conditions are usually defined as a temperature greater than about 250 °F (121 °C) and a pore pressure greater than about 0.8 psi/ft (15.3 lb/gal). In addition, the liquid weighting agents of the invention, when used in offshore applications, need low-temperature thermal stability because temperatures at the sea floor can be around 5 °C.

It is often useful to include one or more optional additives with the liquid weighting agents, especially in a well fluid, and the inclusion of such additives is within the scope of this invention. Optional additives can include, for example, other weighting agents, corrosion inhibitors, lubricants, pH control additives, surfactants, and/or solvents.

Preferred types of optional additives in the practice of this invention are unsaturated diesters and saturated triesters that do not contain bromine. When the additive is an unsaturated diester, the unsaturated diester has at least eight carbon atoms and is represented by the formula

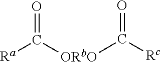

where R^{a} and R^{c} are hydrocarbyl groups, and R^{b} is an alkene-containing moiety. R^{a} and R^{c} can be straight chain, branched, cyclic, or aromatic; preferably the hydrocarbyl group is a straight chain. Suitable hydrocarbyl groups include methyl, ethyl, n-propyl, 2-propyl, n-butyl, pentyl, cyclopentyl, hexyl, and cyclohexyl; ethyl and n-propyl are preferred. R^{b} preferably has four to about eight carbon atoms, more preferably four to about six carbon atoms; suitable alkene-containing moieties include 2-butene, 2-pentene, 2-hexene, 3-hexene, 2-octene 3-octene, and 4-octene; preferred alkene-containing moieties include 2-butuene.

When the additive is a saturated triester, the saturated triester has at least seven carbon atoms and is represented by the formula

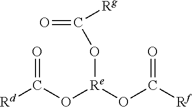

where R^{d}, R^{e}, R^{f}, and R^{g} are hydrocarbyl groups. R^{d}, R^{f}, and R^{g} can be straight chain, branched, or cyclic; preferably the hydrocarbyl group is a straight chain. Suitable hydrocarbyl groups include methyl, ethyl, n-propyl, 2-propyl, n-butyl, pentyl, cyclopentyl, hexyl, cyclohexyl, and phenyl; ethyl and n-propyl are preferred. R^{e} can be branched or cyclic, or aromatic; preferably R^{e} is a saturated branched chain. Suitable hydrocarbyl groups include propyl, butyl, 2-pentyl, 3-pentyl, cyclopentyl, 2-hexyl, 3-hexyl, and cyclohexyl; 2-propyl is a preferred group.

Suitable unsaturated diesters that do not contain bromine include but-2-ene-1,4-diyl diacetate, but-2-ene-1,4-diyl bis(propanoate), but-2-ene-1,4-diyl bis(butyrate), but-2-ene-1,4-diyl bis(pentanoate), pent-2-ene-1,5-diyl diacetate, pent-3-ene-1,5-diyl diacetate, pent-3-ene-1,5-diyl bis(propanoate); preferred is but-2-ene-1,4-diyl bis(butyrate). Suitable saturated triesters that do not contain bromine include propane-1,2,3-triyl triacetate, propane-1,2,3-triyl propanoate, propane-1,2,3-triyl tributyrate; preferred is propane-1,2,3-triyl tributyrate. Combinations of any two or more unsaturated diesters and/or saturated triesters can be used in the practice of the invention.

Processes for forming mixtures of a liquid weighting agent and an oil, which oil can be a paraffin, mineral, olefinic, or ester oil, comprise combining the oil and the liquid weighting agent in any order. For example, an oil and the liquid weighting agent(s) can be added sequentially, or they can be introduced to a vessel or location simultaneously.

Any optional additives that are included can be combined with the liquid weighting agents in any convenient manner. When the liquid weighting agent is in a mixture with an oil, optional additives can be added before, during, and/or after formation of the mixture of an oil and the liquid weighting agent.

In some embodiments of the invention, a mixture of an oil and one or more liquid weighting agents of the invention is solids-free.

Although mixtures comprising liquid weighting agents are preferably solids-free, in some instances, one or more liquid weighting agents of the invention can be part of a mixture containing one or more solids, for example a solid weighting agent such as barite, if desired. In these instances, the use of a liquid weighting agent of the invention will reduce the amount of solid weighting agent needed to achieve the desired density.

In some well fluids, the presence of some solids is not disadvantageous. Calcium carbonate is often present as a suspension in a well fluid. The calcium carbonate often forms a filter cake that prevents the drilling fluid from entering the formation. After drilling, an acid treatment can be used to remove the calcium carbonate filter cake.

One more liquid weighting agents of the invention can be combined with an oil-based well fluid. The oil-based well fluid can be a drilling fluid; completion fluids including but not limited to a packer fluid, a screen running fluid, or a carrier fluid; a (reservoir) drill-in fluid; a tractor fluid; a camera fluid; or workover fluids including but not limited to a coiled tubing drilling fluid. The liquid weighting agents of the invention can be combined with the well fluid alone or in admixture with one or more other additives and/or in a mixture with an oil.

The liquid weighting agents of the invention can be used as secondary fluids (“kill fluids”) to minimize or relieve sustained casing pressure in a wellbore annulus. The term “annular fluid” refers to the first fluid, which is present in the wellbore annulus before the second fluid is introduced. The second fluid can be introduced at the wellhead or below the surface of the annular fluid; subsurface introduction of the kill fluid is generally preferred. For treating sustained casing pressure, immiscibility refers to the non-mixing or substantial non-mixing of the liquid weighting agents and the (aqueous) annular fluid. See in this connection International Publication WO 2015/100004.

The liquid weighting agents of this invention can be used as additives for hydraulic fluids. Preferably, the liquid weighting agent of the invention is about 35 wt % or more relative to the total weight of the hydraulic fluid and the liquid weighting agent of the invention. In terms of bromine content, there is about 20 wt % to about 70 wt % bromine relative to the total weight of the hydraulic fluid and the liquid weighting agent of the invention. Other additives, such as antioxidants, lubricity agents, and metal deactivators, may also be present in the hydraulic fluid.

The molecule 2,3,4,5-tetrabromo-2,4-hexadiene is a new composition of matter. The tribromocumene mixtures of the invention comprising 2,4,5-tribromocumene and one or more unsubstituted tribromobenzenes, wherein 2,4,5-tribromocumene is about 70 wt % or more relative to the total weight of the tribromocumene mixture, and the unsubstituted tribromobenzenes are about 5 wt % or more, relative to the total weight of the mixture are also new compositions of matter.

The following examples are presented for purposes of illustration, and are not intended to impose limitations on the scope of this invention.

### EXAMPLE 1

### Synthesis of 1,2-dibromo-1-hexene

To a flask containing dichloromethane (100 mL), 1-hexyne (0.46 mol) was added and dissolved while stirring. Br₂ (67 g, 0.42 mol) was added to the 1-hexyne solution during 1 hour and 35 minutes while maintaining the temperature below 3.7 °C. The reaction mixture was then stirred for 3 hours at −1 °C, and then allowed to warm to room temperature. The reaction mixture was washed with aqueous sodium sulfite and then with water. The phases were allowed to separate, dichloromethane was removed from the organic layer under vacuum (~60 torr), to obtain 91 grams of the product as a liquid with a density of 1.61 g/mL. The 1,2-dibromo-1-hexene was miscible with a base oil in a 50:50 vol/vol mixture with a base oil at −18 °C.

### EXAMPLE 2

### Synthesis of 2,3-dibromo-2-butene

To a flask containing dichloromethane (100 mL), 2-butyne (25 g, 0.46 mol) was added and dissolved while stirring. Br₂ (67 g, 0.42 mol) was added to the 2-butyne solution during 1 hour and 35 minutes while maintaining the temperature below 3.7 °C. The reaction mixture was then stirred for 3 hours at −1 °C, and then allowed to warm to room temperature. The reaction mixture was washed with aqueous sodium sulfite and then with water. The phases were allowed to separate, dichloromethane was removed from the organic layer under vacuum (~60 torr), to obtain 91 grams of the product as a liquid with a density of 1.78 g/mL. A 50:50 vol/vol mixture of 2,3-dibromobutene with a base oil had not phase separated and was solids-free after one week at −18 °C.

### EXAMPLE 3

### Synthesis of 2,3,4,5-tetrabromo-2,4-hexadiene

To a flask containing dichloromethane (100 mL), 2,4-hexadiyne (19.3 g, 0.25 mol) was added and dissolved, and then the solution was cooled to 0 °C while stirring. Br₂ (75 g, 0.47 mol) was added over 1.5 hours while maintaining the temperature of the reaction mixture below 3 °C. After stirring the reaction mixture at 2 °C for 3 hours, the reaction mixture was allowed to warm to room temperature. The reaction mixture was washed with aqueous sodium sulfite and then with water. The phases were allowed to separate, and the organic phase was dried with MgSO₄ and filtered. The liquid from the filtration was subjected to vacuum to remove the dichloromethane was dried over magnesium sulfate and filtered, to obtain 96 grams of the product as a liquid with a density of 2.13 g/mL. The 2,3,4,5-tetrabromo-2,4-hexadiene product was combined with a base oil in a 50:50 volume/volume mixture; the mixture was miscible at room temperature, and no solid formation or phase separation was observed after one week at −18 °C.

### EXAMPLE 4

### Synthesis of tribromocumene (2,4,5-tribromocumene)

Under an inert atmosphere, an organic solvent (e.g., a bromochloromethane and dibromomethane mixture, or dibromomethane alone) and isopropylbenzene (cumene) were charged to a flask, followed by a catalytic amount of anhydrous aluminum chloride (AlCl₃, 0.3 wt % to 0.5 wt % relative to cumene). The temperature of the mixture in the flask was lowered to −3 °C, and while keeping the temperature at −3 °C to −1 °C and stirring, Br₂ (slightly in excess of 3 moles per mole of cumene) was added dropwise to the mixture over a period of time. After the bromine addition was completed, the reaction mixture was stirred for another period of time, after which the reaction mixture was allowed to warm to room temperature. After the reaction mixture reached room temperature, the reaction mixture was washed with water, the phases were allowed to separate, and the organic phase was subjected to vacuum to remove the organic solvent. The tribromocumene product mixture had a density of 2 g/mL and a viscosity of 6.8 cP at 40 °C. The components of the mixture are summarized in Table 1.

TABLE 1

| Isomer | Amount | |
|---|---|---|
| 2,4,5-tribromocumene | 71.16 | wt % |
| 2,3,5-tribromocumene | 3.74 | wt % |
| 3,4,5-tribromocumene | 1.45 | wt % |
| 2,3,4,5-tetrabromocumene | 8.26 | wt % |
| 1,2,4,5-tetrabromobenzene | 0.97 | wt % |
| 1,2,4-tribromobenzene | 6.84 | wt % |
| 1,3,5-tribromobenzene | 1.19 | wt % |

### EXAMPLE 4A

### Additional Syntheses of tribromocumene (2,4,5-tribromocumene)

Another series of four (4) separate runs for forming a 2,4,5-tribromocumene product mixture were conducted using the same procedure as set forth in Example 4. The starting material amounts, resulting product yields and characterizations are summarized in the following Table 1A.

TABLE 1A

| Run | 1 | 2 | 3 | 4 |
|---|---|---|---|---|
| Cumene (g) | 775 | 800 | 1086.3 | 948 |
| Bromine (g, soln) | 3165 | 3262 | 4430 | 3875 |
| AlCl₃ | 33 | 33 | 43 | 37.90 |
| Bromochloromethane (g) | 2129.6 | 2088 | 0 | 0 |
| Dibromomethane (g) | 532.4 | 522 | 0 | 3611 |
| Br₂ Feed Time (hours) | 3.5 | 3.5 | 3.8 | 2.7 |
| Br₂ Feed Rate (g/hr) | 904 | 932 | 1166 | 1435 |
| Actual Yield (g) | 2226 | 2330 | 3076 | 2676 |
| Density | 2 | 2 | 1.97 | 2 |
| 2,4,5-tribromocumene (GC | 76.58 | 83.28 | 67.57 | 74.12 |
| area sum %) | | | | |
| 2,3,5-tribromocumene | 4.4 | 4.79 | 3.16 | 3.44 |
| (GC area sum %) | | | | |
| 3,4,5-tribromocumene (GC | 0.97 | 0.72 | 2.30 | 2.26 |
| area sum %) | | | | |
| 2,3,4,5-tetrabromocumene | 9.36 | 6.53 | 20.54 | 12.64 |
| (GC area sum %) | | | | |
| 1,2,4-tribromobenzene | 6.28 | 4.24 | 3.57 | 3.92 |
| (GC area sum %) | | | | |
| 1,3,5-tribromobenezene | 0 | 0 | 0 | 0 |
| (GC area sum %) | | | | |
| Dibromocumenes | 0 | 0 | 0.35 | 0 |
| (GC area sum %) | | | | |
| Diisopropyl species | 0.41 | 0 | 2.00 | 1.17 |
| (GC area sum %) | | | | |

### TESTING PROCEDURES

Admixture preparation. To a 600 mL beaker were added a synthetic oil fluid (Saraline® 185V), and a liquid weighting agent of the invention. An overhead lab mixer (Silverson, model L5M-A) is lowered into the liquid and slowly turned up to 4000 rpm. Various ingredients were then added sequentially, stirring for a period of time after each addition. The mixture was transferred to a stand mixer (Hamilton Beach), usually before the addition of calcium carbonate. When a liquid polymeric viscosifier was added, ice cubes were held against the outside of the Hamilton Beach stand mixer during the mixing of the liquid polymeric viscosifier to control the temperature because the standard procedure requires testing at room temperature (20 to 25 °C), and the mixing procedure usually increased the temperature to over 110 °F (43.3 °C). Sample components, amounts thereof, mixer type, and mixing time after each addition are summarized in the Tables below.

Admixture testing. The American Petroleum Institute Recommended Practice 13B-2 (API RP 13B-2) testing procedures for field testing oil-based drilling fluids were followed to obtain properties of the mixture formed as described above. The sample was then hot-rolled overnight to simulate heat aging of the sample in actual use. The properties of the sample were then tested again using the same testing procedures.

The components and testing results for a tribromocumene mixture are summarized in Tables 2A-2B below. The hot-rolling temperature was 177 °C.

TABLE 2A

| Substance | Amount | | Mixing time | | Mixer |
|---|---|---|---|---|---|
| tribromocumene | 170 | mL | — | | — |
| C₉₋₂₁ hydrocarbons (synthetic oil fluid) | 55 | mL | — | | — |
| (Saraline ® 185V) | | | | | |
| organic derivative of hectorite clay | 2.5 | g | 25 | min. | overhead |
| (Bentone ® 38) | | | | | |
| Tall oil distillates (TCM MUL P 350) | 8 | g | 10 | min. | overhead |
| Tall oil distillates (TCM MUL S 350) | 4 | g | 10 | min. | overhead |
| Dipropylene glycol monomethyl ether | 1 | g | 10 | min. | overhead |
| (Thixatrol ® DW50) | | | | | |
| Ca(OH)₂ (lime) | 5.5 | g | 10 | min. | overhead |
| CaBr₂ (aq., 14.2 ppg) | 119 | g | 10 | min. | overhead |
| H₂O | 6 | g | 10 | min. | overhead |
| CaCO₃ (Omya ® 04) | 45 | g | 3 | min. | stand |
| CaCO₃ (Omya ® 08) | 30 | g | 3 | min. | stand |
| CaCO₃ (Omya ® 15) | 10 | g | 3 | min. | stand |
| Ethoxylated alkylether sulfate, Na salt | 0.5 | g | 3 | min. | stand |
| (liquid polymeric viscosifier; Pexovis ® 553) | | | | | |

TABLE 2B

| | | Run | | | |
|---|---|---|---|---|---|
| | | 1 | 2 | 3 | 4 |
| | | Before hot-rolling | | After hot-rolling | |
| | | Temperature | | | |
| | | 74 °F | 148 °F | 81 °F | 146 °F |
| | | (23.3 °C) | (64.4 °C) | (27.2 °C) | (63.3 °C) |
| Stirrer | Shear rate, | Shear | Shear | Shear | Shear |
| speed, RPM | Hz | stress | stress | stress | stress |
| 600 | 1022 | 226 | 77 | 185 | 107 |
| 300 | 511 | 140 | 44 | 121 | 75 |
| 200 | 341 | 105 | 31 | 95 | 61 |
| 100 | 170 | 66 | 19 | 66 | 45 |
| 6 | 10 | 34 | 9 | 27 | 23 |
| 3 | 5 | 32 | 8 | 25 | 22 |

| | | | | |
|---|---|---|---|---|
| Gel strength at 10 sec., | 57 | 17 | 30 | 27 |
| lb/100 ft² | | | | |
| Gel strength at 10 min., | 67 | 20 | 47 | 31 |
| lb/100 ft² | | | | |
| Electrical stability, V* | 612 | 647 | 738 | 923 |
| Plastic viscosity, cP | 86 | 33 | 64 | 32 |
| Yield Point, lb/100 ft² | 54 | 11 | 57 | 43 |

*Reported values are an average of two runs.

Another batch of the same components with the same mixing parameters as in Table 2A was prepared, and results for this second tribromocumene mixture are summarized in Table 2C below. The hot-rolling temperature was 177 °C.

TABLE 2C

| | | Run | | | |
|---|---|---|---|---|---|
| | | 1 | 2 | 3 | 4 |
| | | Before hot-rolling | | After hot-rolling | |
| | | Temperature | | | |
| | | 78 °F | 149 °F | 73 °F | 151 °F |
| | | (25.6 °C) | (65.0 °C) | (22.8 °C) | (66.1 °C) |
| Stirrer | Shear rate, | Shear | Shear | Shear | Shear |
| speed, RPM | Hz | stress | stress | stress | stress |
| 600 | 1022 | 209 | 91 | 207 | 101 |
| 300 | 511 | 121 | 55 | 134 | 69 |
| 200 | 341 | 92 | 39 | 104 | 55 |
| 100 | 170 | 60 | 25 | 71 | 39 |
| 6 | 10 | 27 | 12 | 27 | 20 |
| 3 | 5 | 25 | 11 | 24 | 19 |

| | | | | |
|---|---|---|---|---|
| Gel strength at 10 sec., | 52 | 23 | 29 | 25 |
| lb/100 ft² | | | | |
| Gel strength at 10 min., | 65 | 26 | 44 | 27 |
| lb/100 ft² | | | | |
| Electrical stability, V* | 661 | 774 | 723 | 910 |
| Plastic viscosity, cP | 88 | 36 | 73 | 32 |
| Yield Point, lb/100 ft² | 33 | 19 | 61 | 37 |

*Reported values are an average of two runs.

Components and testing results for another tribromocumene mixture are summarized in Tables 3A-3B below. The static aging temperature was 121 °C, and the aging was for 48 hours instead of overnight.

TABLE 3A

| Substance | Amount | | Mixing time | | Mixer |
|---|---|---|---|---|---|
| tribromocumene | 170 | mL | — | | — |
| C₉₋₂₁ hydrocarbons (synthetic oil fluid) | 55 | mL | — | | — |
| (Saraline ® 185V) | | | | | |
| organic derivative of hectorite clay | 2.5 | g | 25 | min. | overhead |
| (Bentone ® 38) | | | | | |
| Tall oil distillates (TCM MUL P 350) | 8 | g | 10 | min. | overhead |
| Tall oil distillates (TCM MUL S 350) | 4 | g | 10 | min. | overhead |
| Dipropylene glycol monomethyl ether | 1 | g | 10 | min. | overhead |
| (Thixatrol ® DW50) | | | | | |
| Ca(OH)₂ (lime) | 5.5 | g | 10 | min. | overhead |
| CaBr₂ (aq., 14.2 ppg) | 119 | g | 10 | min. | overhead |
| H₂O | 6 | g | 10 | min. | overhead |
| CaCO₃ (Omya ® 04) | 4.5 | g | 3 | min. | stand |
| CaCO₃ (Omya ® 08) | 3 | g | 3 | min. | stand |
| CaCO₃ (Omya ® 15) | 1 | g | 3 | min. | stand |
| Ethoxylated alkylether sulfate, Na salt | 0.5 | g | 13 | min. | stand |
| (liquid polymeric viscosifier; Pexovis ® 553) | | | | | |

TABLE 3B

| | | Run | | | |
|---|---|---|---|---|---|
| | | 1 | 2 | 3 | 4 |
| | | Before static aging | | After static aging | |
| | | Temperature | | | |
| | | 77 °F | 150 °F | 72 °F | 150 °F |
| | | (25.0 °C) | (65.6 °C) | (22.2 °C) | (65.6 °C) |
| Stirrer | Shear rate, | Shear | Shear | Shear | Shear |
| speed, RPM | Hz | stress | stress | stress | stress |
| 600 | 1022 | 125 | 44 | 119 | 48 |
| 300 | 511 | 74 | 25 | 72 | 29 |
| 200 | 341 | 54 | 18 | 54 | 22 |
| 100 | 170 | 33 | 12 | 35 | 15 |
| 6 | 10 | 10 | 5 | 11 | 7 |
| 3 | 5 | 9 | 5 | 10 | 6 |

| | | | | |
|---|---|---|---|---|
| Gel strength at 10 sec., | 16 | 7 | 12 | 7 |
| lb/100 ft² | | | | |
| Gel strength at 10 min., | 21 | 8 | 17 | 8 |
| lb/100 ft² | | | | |
| Electrical stability, V* | 400 | 554 | 529 | 540 |
| Plastic viscosity, cP | 51 | 19 | 47 | 19 |
| Yield Point, lb/100 ft² | 23 | 6 | 25 | 10 |

*Reported values are an average of two runs.

Components and testing results for another tribromocumene mixture are summarized in Tables 4A-4B below. The hot-rolling temperature was 149 °C.

TABLE 4A

| Substance | Amount | | Mixing time | | Mixer |
|---|---|---|---|---|---|
| tribromocumene | 215 | mL | — | | — |
| C₁₆₋₁₈ hydrocarbons (synthetic oil fluid) | 70 | mL | — | | — |
| (AlphaPlus ® C16/18 Internal Olefin) | | | | | |
| organic derivative of bentonite clay | 4 | g | 20 | min. | overhead |
| (Bentone ® 150) | | | | | |
| Hydrotreated distillate and tall oil | 6 | g | 10 | min. | overhead |
| (AES MUL X) | | | | | |
| Hydrotreated distillate and tall oil | 3 | g | 10 | min. | overhead |
| (AES MUL X) | | | | | |
| Dipropylene glycol monomethyl ether | 1 | g | 10 | min. | overhead |
| (Thixatrol ® DW50) | | | | | |
| organic derivative of hectorite clay | 2.5 | g | 20 | min. | overhead |
| (Bentone ® 38) | | | | | |
| CaCl₂ * 2H₂O | 14.6 | g | 10 | min. | overhead |
| H₂O | 25.5 | g | 10 | min. | overhead |
| Ca(OH)₂ (lime) | 5 | g | 10 | min. | overhead |
| CaCO₃ (Omya ® 04) | 15 | g | 6 | min. | stand |
| CaCO₃ (Omya ® 08) | 15 | g | 6 | min. | stand |
| CaCO₃ (Omya ® 15) | 7 | g | 6 | min. | stand |
| CaCO₃ (Omya ® Titan 200) | 5 | g | 6 | min. | stand |
| Ethoxylated alkylether sulfate, Na salt | 0.5 | g | 13 | min. | stand |
| (liquid polymeric viscosifier; Pexovis ® 553) | | | | | |

TABLE 4B

| | | Run | | | |
|---|---|---|---|---|---|
| | | 1 | 2 | 3 | 4 |
| | | Before hot-rolling | | After hot-rolling | |
| | | Temperature | | | |
| | | 72 °F | 147 °F | 72 °F | 145 °F |
| | | (22.2 °C) | (63.9 °C) | (22.2 °C) | (62.8 °C) |
| Stirrer | Shear rate, | Shear | Shear | Shear | Shear |
| speed, RPM | Hz | stress | stress | stress | stress |
| 600 | 1022 | 116 | 55 | 124 | 57 |
| 300 | 511 | 76 | 36 | 81 | 39 |
| 200 | 341 | 59 | 28 | 63 | 30 |
| 100 | 170 | 37 | 18 | 41 | 21 |
| 6 | 10 | 14 | 8 | 15 | 10 |
| 3 | 5 | 13 | 7 | 14 | 9 |

| | | | | |
|---|---|---|---|---|
| Gel strength at 10 sec., | 16 | 9 | 18 | 14 |
| lb/100 ft² | | | | |
| Gel strength at 10 min., | 24 | 13 | 35 | 20 |
| lb/100 ft² | | | | |
| Electrical stability, V* | 1549 | 1909 | >2000 | >2000 |
| Plastic viscosity, cP | 40 | 19 | 43 | 18 |
| Yield Point, lb/100 ft² | 36 | 17 | 38 | 21 |

*Reported values are an average of two runs.

Components and testing results for another tribromocumene mixture are summarized in Tables 5A-5B below. The hot-rolling temperature was 149 °C.

TABLE 5A

| Substance | Amount | | Mixing time | | Mixer |
|---|---|---|---|---|---|
| tribromocumene | 251 | mL | — | | — |
| organic derivative of bentonite clay | 2 | g | 30 | min. | overhead |
| (Bentone ® 150) | | | | | |
| organic derivative of hectorite clay | 1.5 | g | 30 | min. | overhead |
| (Bentone ® 38) | | | | | |
| Hydrotreated distillate and tall oil | 6 | g | 10 | min. | overhead |
| (AES MUL X) | | | | | |
| Hydrotreated distillate and tall oil | 6 | g | 10 | min. | overhead |
| (AES MUL X) | | | | | |
| Dipropylene glycol monomethyl ether | 1 | g | 10 | min. | overhead |
| (Thixatrol ® DW50) | | | | | |
| CaCl₂ * 2H₂O | 13 | g | 10 | min. | overhead |
| H₂O | 22.5 | g | 10 | min. | overhead |
| Ca(OH)₂ (lime) | 5 | g | 10 | min. | overhead |
| CaCO₃ (Omya ® 04) | 15 | g | 2 | min. | stand |
| CaCO₃ (Omya ® 08) | 15 | g | 2 | min. | stand |
| CaCO₃ (Omya ® 15) | 7 | g | 2 | min. | stand |
| CaCO₃ (Omya ® Titan 200) | 5 | g | 2 | min. | stand |
| BaSO₄ (barite) | 150 | g | 2 | min. | |
| Ethoxylated alkylether sulfate, Na salt | 1 | g | 10 | min. | stand |
| (liquid polymeric viscosifier; Pexovis ® 553) | | | | | |

TABLE 5B

| | | Run | | | |
|---|---|---|---|---|---|
| | | 1 | 2 | 3 | 4 |
| | | Before hot-rolling | | After hot-rolling | |
| | | Temperature | | | |
| | | 71 °F | 147 °F | 68 °F | 148 °F |
| | | (21.7 °C) | (63.9 °C) | (20 °C) | (64.4 °C) |
| Stirrer | Shear rate, | Shear | Shear | Shear | Shear |
| speed, RPM | Hz | stress | stress | stress | stress |
| 600 | 1022 | 256 | 76 | 263 | 64 |
| 300 | 511 | 153 | 46 | 152 | 41 |
| 200 | 341 | 115 | 34 | 112 | 29 |
| 100 | 170 | 70 | 21 | 66 | 18 |
| 6 | 10 | 21 | 9 | 16 | 7 |
| 3 | 5 | 19 | 8 | 13 | 6 |

| | | | | |
|---|---|---|---|---|
| Gel strength at 10 sec., | 25 | 11 | 18 | 9 |
| lb/100 ft² | | | | |
| Gel strength at 10 min., | 35 | 15 | 29 | 14 |
| lb/100 ft² | | | | |
| Electrical stability, V* | >2000 | >2000 | 1444 | 1274 |
| Plastic viscosity, cP | 103 | 30 | 111 | 23 |
| Yield Point, lb/100 ft² | 50 | 16 | 41 | 18 |

*Reported values are an average of two runs.

Components and testing results for another tribromocumene mixture are summarized in Tables 6A-6B below. The hot-rolling temperature was 149 °C.

TABLE 6A

| Substance | Amount | | Mixing time | | Mixer |
|---|---|---|---|---|---|
| tribromocumene | 250 | mL | — | | — |
| organic derivative of bentonite clay | 2 | g | 30 | min. | overhead |
| (Bentone ® 150) | | | | | |
| organic derivative of hectorite clay | 1.5 | g | 30 | min. | overhead |
| (Bentone ® 38) | | | | | |
| Hydrotreated distillate and tall oil | 6 | g | 10 | min. | overhead |
| (AES MUL X) | | | | | |
| Hydrotreated distillate and tall oil | 6 | g | 10 | min. | overhead |
| (AES MUL X) | | | | | |
| Dipropylene glycol monomethyl ether | 1 | g | 10 | min. | overhead |
| (Thixatrol ® DW50) | | | | | |
| CaCl₂ * 2H₂O | 13 | g | 10 | min. | overhead |
| H₂O | 22.5 | g | 10 | min. | overhead |
| Ca(OH)₂ (lime) | 5 | g | 10 | min. | overhead |
| CaCO₃ (Omya ® 04) | 15 | g | 2 | min. | stand |
| CaCO₃ (Omya ® 08) | 15 | g | 2 | min. | stand |
| CaCO₃ (Omya ® 15) | 7 | g | 2 | min. | stand |
| CaCO₃ (Omya ® Titan 200) | 5 | g | 2 | min. | stand |
| Mn₃O₄ | 150 | g | 2 | min. | stand |
| Ethoxylated alkylether sulfate, Na salt | 0.5 | g | 10 | min. | stand |
| (liquid polymeric viscosifier; Pexovis ® 553) | | | | | |

TABLE 6B

| | | Run | | | |
|---|---|---|---|---|---|
| | | 1 | 2 | 3 | 4 |
| | | Before hot-rolling | | After hot-rolling | |
| | | Temperature | | | |
| | | 71 °F | 147 °F | 68 °F | 148 °F |
| | | (21.7 °C) | (63.9 °C) | (20 °C) | (64.4 °C) |
| Stirrer | Shear rate, | Shear | Shear | Shear | Shear |
| speed, RPM | Hz | stress | stress | stress | stress |
| 600 | 1022 | 261 | 87 | 274 | 76 |
| 300 | 511 | 164 | 57 | 160 | 44 |
| 200 | 341 | 122 | 43 | 117 | 34 |
| 100 | 170 | 75 | 28 | 68 | 21 |
| 6 | 10 | 21 | 11 | 15 | 7 |
| 3 | 5 | 19 | 10 | 12 | 6 |

| | | | | |
|---|---|---|---|---|
| Gel strength at 10 sec., | 24 | 14 | 15 | 14 |
| lb/100 ft² | | | | |
| Gel strength at 10 min., | 34 | 19 | 27 | 18 |
| lb/100 ft² | | | | |
| Electrical stability, V* | 1936 | 1581 | 1390 | 1800 |
| Plastic viscosity, cP | 97 | 30 | 114 | 32 |
| Yield Point, lb/100 ft² | 67 | 27 | 46 | 12 |

*Reported values are an average of two runs.

Another set of components and testing results for tribromocumene are summarized in Tables 7A-7P below. The relative amounts of tribromocumene and the synthetic oil fluid were varied in these runs. The hot-rolling temperature was 105 °C, 121 °C, 149 °C, or 177 °C, as specified in the tables below.

TABLE 7A

| Substance | Amount | | Mixing time | | Mixer |
|---|---|---|---|---|---|
| tribromocumene | 55 to 113 | mL | — | | — |
| C₁₆₋₂₂ hydrocarbons (synthetic oil fluid) | 113 to 170 | mL | — | | — |
| (Saraline ® 185V) | | | | | |
| organic derivative of bentonite clay | 4 | g | 20 | min. | overhead |
| (Bentone ® 150) | | | | | |
| Hydrotreated distillate and tall oil | 6 | g | 10 | min. | overhead |
| (AES MUL X) | | | | | |
| Hydrotreated distillate and tall oil | 3 | g | 10 | min. | overhead |
| (AES MUL X) | | | | | |
| Dipropylene glycol monomethyl ether | 1 | g | 10 | min. | overhead |
| (Thixatrol ® DW50) | | | | | |
| organic derivative of hectorite clay | 2.5 | g | 20 | min. | overhead |
| (Bentone ® 38) | | | | | |
| CaCl₂ * 2H₂O | 14.6 | g | 10 | min. | overhead |
| H₂O | 25.5 | g | 10 | min. | overhead |
| Ca(OH)₂ (lime) | 5 | g | 10 | min. | overhead |
| CaCO₃ (Omya ® 04) | 15 | g | 6 | min. | stand |
| CaCO₃ (Omya ® 08) | 15 | g | 6 | min. | stand |
| CaCO₃ (Omya ® 15) | 7 | g | 6 | min. | stand |
| CaCO₃ (Omya ® Titan 200) | 5 | g | 6 | min. | stand |
| Ethoxylated alkylether sulfate, Na salt | 0.5 | g | 13 | min. | stand |
| (liquid polymeric viscosifier; Pexovis ® 553) | | | | | |

TABLE 7B
112.5 mL tribromocumene
112.5 mL synthetic oil fluid
Hot-rolling temperature: 105 °C

| | | Run | | | |
|---|---|---|---|---|---|
| | | 1 | 2 | 3 | 4 |
| | | Before hot-rolling | | After hot-rolling | |
| | | Temperature | | | |
| | | 74 °F | 116 °F | 74 °F | 120 °F |
| | | (23.3 °C) | (46.7 °C) | (23.3 °C) | (48.9 °C) |
| Stirrer | Shear rate, | Shear | Shear | Shear | Shear |
| speed, RPM | Hz | stress | stress | stress | stress |
| 600 | | 128 | 75 | 151 | 105 |
| 300 | | 81 | 47 | 101 | 67 |
| 200 | | 62 | 37 | 77 | 53 |
| 100 | | 42 | 26 | 46 | 37 |
| 6 | | 16 | 13 | 16 | 19 |
| 3 | | 14 | 12 | 14 | 17 |

| | | | | |
|---|---|---|---|---|
| Gel strength at 10 sec., | 18 | 17 | 20 | 20 |
| lb/100 ft² | | | | |
| Electrical stability, V* | 676 | 811 | 788 | 906 |
| Plastic viscosity, cP | 47 | 28 | 50 | 38 |
| Yield Point, lb/100 ft² | 34 | 19 | 51 | 29 |

*Reported values are an average of two runs.

TABLE 7C
168.75 mL tribromocumene
56.25 mL synthetic oil fluid
Hot-rolling temperature: 105 °C

| | Run | | | |
|---|---|---|---|---|
| | 1 | 2 | 3 | 4 |
| | Before hot-rolling | | After hot-rolling | |
| | Temperature | | | |
| | 75 °F | 122 °F | 68 °F | 115 °F |
| | (23.9 °C) | (50 °C) | (20 °C) | (46.1 °C) |
| | Shear | Shear | Shear | Shear |
| Stirrer speed, RPM | stress | stress | stress | stress |
| 600 | 202 | 123 | 242 | 129 |
| 300 | 135 | 85 | 145 | 84 |
| 200 | 109 | 71 | 108 | 69 |
| 100 | 78 | 54 | 66 | 49 |
| 6 | 37 | 31 | 24 | 29 |
| 3 | 35 | 30 | 21 | 27 |
| Gel strength at 10 sec., | 39 | 37 | 26 | 30 |
| lb/100 ft² | | | | |
| Electrical stability, V* | 1271 | — | 1087 | 1159 |
| Plastic viscosity, cP | 67 | 38 | 97 | 45 |
| Yield Point, lb/100 ft² | 68 | 47 | 48 | 39 |

*Reported values are an average of two runs.

TABLE 7D
112.5 mL tribromocumene
112.5 mL synthetic oil fluid
Hot-rolling temperature: 105 °C

| | Run | | | |
|---|---|---|---|---|
| | 1 | 2 | 3 | 4 |
| | Before hot-rolling | | After hot-rolling | |
| | Temperature | | | |
| | 79 °F | 118 °F | 79 °F | 120 °F |
| | (26.1 °C) | (47.8 °C) | (26.1 °C) | (48.9 °C) |
| | Shear | Shear | Shear | Shear |
| Stirrer speed, RPM | stress | stress | stress | stress |
| 600 | 99 | 55 | 115 | 63 |
| 300 | 59 | 35 | 66 | 38 |
| 200 | 44 | 26 | 48 | 29 |
| 100 | 28 | 17 | 29 | 19 |
| 6 | 8 | 7 | 8 | 7 |
| 3 | 7 | 7 | 7 | 6 |
| Gel strength at 10 sec., | 10 | 9 | 10 | 9 |
| lb/100 ft² | | | | |
| Gel strength at 10 min., | 18 | 15 | 17 | 15 |
| lb/100 ft² | | | | |
| Electrical stability, V* | 273 | 533 | 517 | 514 |
| Plastic viscosity, cP | 40 | 23 | 49 | 25 |
| Yield Point, lb/100 ft² | 19 | 12 | 17 | 13 |

*Reported values are an average of two runs.

TABLE 7E
168.75 mL tribromocumene
56.25 mL synthetic oil fluid
Hot-rolling temperature: 121 °C

| | Run | | | |
|---|---|---|---|---|
| | 1 | 2 | 3 | 4 |
| | Before hot-rolling | | After hot-rolling | |
| | Temperature | | | |
| | 79 °F | 148 °F | 75 °F | 149 °F |
| | (26.1 °C) | (64.4 °C) | (23.9 °C) | (65 °C) |
| | Shear | Shear | Shear | Shear |
| Stirrer speed, RPM | stress | stress | stress | stress |
| 600 | 130 | 58 | 138 | 58 |
| 300 | 72 | 33 | 79 | 34 |
| 200 | 51 | 25 | 58 | 25 |
| 100 | 29 | 15 | 36 | 16 |
| 6 | 5 | 6 | 10 | 7 |
| 3 | 4 | 5 | 9 | 6 |
| Gel strength at 10 sec., | 6 | 9 | 11 | 9 |
| lb/100 ft² | | | | |
| Gel strength at 10 min., | 17 | 18 | 20 | 18 |
| lb/100 ft² | | | | |
| Electrical stability, V* | 923 | 1161 | 785 | 544 |
| Plastic viscosity, cP | 58 | 25 | 59 | 24 |
| Yield Point, lb/100 ft² | 14 | 8 | 20 | 10 |

*Reported values are an average of two runs.

TABLE 7F
168.75 mL tribromocumene
56.25 mL synthetic oil fluid
Hot-rolling temperature: 149 °C

| | Run | | | |
|---|---|---|---|---|
| | 1 | 2 | 3 | 4 |
| | Before hot-rolling | | After hot-rolling | |
| | Temperature | | | |
| | 77 °F | 149 °F | 74 °F | 148 °F |
| | (26.1 °C) | (65 °C) | (23.3 °C) | (64.4 °C) |
| | Shear | Shear | Shear | Shear |
| Stirrer speed, RPM | stress | stress | stress | stress |
| 600 | 130 | 53 | 135 | 53 |
| 300 | 70 | 30 | 73 | 29 |
| 200 | 50 | 22 | 51 | 20 |
| 100 | 28 | 14 | 29 | 12 |
| 6 | 5 | 5 | 5 | 3 |
| 3 | 4 | 4 | 4 | 2 |
| Gel strength at 10 sec., | 6 | 8 | 6 | 4 |
| lb/100 ft² | | | | |
| Gel strength at 10 min., | 14 | 16 | 9 | 9 |
| lb/100 ft² | | | | |
| Electrical stability, V* | 936 | 1138 | 370 | 260 |
| Plastic viscosity, cP | 60 | 23 | 62 | 24 |
| Yield Point, lb/100 ft² | 10 | 7 | 11 | 5 |

*Reported values are an average of two runs.

TABLE 7G
168.75 mL tribromocumene
56.25 mL synthetic oil fluid
Hot-rolling temperature: 149 °C

| | Run | | | |
|---|---|---|---|---|
| | 1 | 2 | 3 | 4 |
| | Before hot-rolling | | After hot-rolling | |
| | Temperature | | | |
| | 79 °F | 146 °F | 73 °F | 146 °F |
| | (26.1 °C) | (63.3 °C) | (22.8 °C) | (63.3 °C) |
| | Shear | Shear | Shear | Shear |
| Stirrer speed, RPM | stress | stress | stress | stress |
| 600 | 132 | 61 | 149 | 51 |
| 300 | 86 | 35 | 80 | 27 |
| 200 | 67 | 26 | 52 | 19 |
| 100 | 45 | 16 | 30 | 11 |
| 6 | 13 | 5 | 5 | 3 |
| 3 | 10 | 4 | 4 | 3 |
| Gel strength at 10 sec., | 11 | 6 | 4 | 5 |
| lb/100 ft² | | | | |
| Gel strength at 10 min., | 16 | 8 | 10 | 10 |
| lb/100 ft² | | | | |
| Electrical stability, V* | 311 | 203 | 214 | 204 |
| Plastic viscosity, cP | 46 | 26 | 69 | 24 |
| Yield Point, lb/100 ft² | 40 | 9 | 11 | 3 |

*Reported values are an average of two runs.

TABLE 7H
168.75 mL tribromocumene
56.25 mL synthetic oil fluid
Hot-rolling temperature: 149 °C

| | Run | | | |
|---|---|---|---|---|
| | 1 | 2 | 3 | 4 |
| | Before hot-rolling | | After hot-rolling | |
| | Temperature | | | |
| | 81 °F | 149 °F | 79 °F | 150 °F |
| | (27.2 °C) | (65 °C) | (26.1 °C) | (65.6 °C) |
| | Shear | Shear | Shear | Shear |
| Stirrer speed, RPM | stress | stress | stress | stress |
| 600 | 206 | 84 | 219 | 116 |
| 300 | 124 | 49 | 142 | 71 |
| 200 | 103 | 35 | 103 | 58 |
| 100 | 67 | 21 | 71 | 41 |
| 6 | 31 | 8 | 32 | 23 |
| 3 | 29 | 8 | 30 | 22 |
| Gel strength at 10 sec., | 54 | 19 | 43 | 32 |
| lb/100 ft² | | | | |
| Gel strength at 10 min., | 74 | 23 | 57 | 33 |
| lb/100 ft² | | | | |
| Electrical stability, V* | 722 | 756 | 931 | 1096 |
| Plastic viscosity, cP | 82 | 35 | 77 | 45 |
| Yield Point, lb/100 ft² | 42 | 14 | 65 | 26 |

*Reported values are an average of two runs.

TABLE 7I
168.75 mL tribromocumene
56.25 mL synthetic oil fluid
Hot-rolling temperature: 177 °C

| | Run | | | |
|---|---|---|---|---|
| | 1 | 2 | 3 | 4 |
| | Before hot-rolling | | After hot-rolling | |
| | Temperature | | | |
| | 78 °F | 149 °F | 73 °F | 151 °F |
| | (25.6 °C) | (65 °C) | (22.8 °C) | (66.1 °C) |
| | Shear | Shear | Shear | Shear |
| Stirrer speed, RPM | stress | stress | stress | stress |
| 600 | 209 | 91 | 207 | 101 |
| 300 | 121 | 55 | 134 | 69 |
| 200 | 92 | 39 | 104 | 55 |
| 100 | 60 | 25 | 71 | 39 |
| 6 | 27 | 12 | 27 | 20 |
| 3 | 25 | 11 | 24 | 19 |
| Gel strength at 10 sec., | 52 | 23 | 29 | 25 |
| lb/100 ft² | | | | |
| Gel strength at 10 min., | 65 | 26 | 44 | 27 |
| lb/100 ft² | | | | |
| Electrical stability, V* | 661 | 774 | 723 | 910 |
| Plastic viscosity, cP | 88 | 36 | 73 | 32 |
| Yield Point, lb/100 ft² | 33 | 19 | 61 | 37 |

*Reported values are an average of two runs.

TABLE 7J
168.75 mL tribromocumene
56.25 mL synthetic oil fluid
Hot-rolling temperature: 177 °C

| | Run | | | |
|---|---|---|---|---|
| | 1 | 2 | 3 | 4 |
| | Before hot-rolling | | After hot-rolling | |
| | Temperature | | | |
| | 74 °F | 148 °F | 81 °F | 146 °F |
| | (23.3 °C) | (64.4 °C) | (27.2 °C) | (63.3 °C) |
| | Shear | Shear | Shear | Shear |
| Stirrer speed, RPM | stress | stress | stress | stress |
| 600 | 226 | 77 | 185 | 107 |
| 300 | 140 | 44 | 121 | 75 |
| 200 | 105 | 31 | 95 | 61 |
| 100 | 66 | 19 | 66 | 45 |
| 6 | 34 | 9 | 27 | 23 |
| 3 | 32 | 8 | 25 | 22 |
| Gel strength at 10 sec., | 57 | 17 | 30 | 27 |
| lb/100 ft² | | | | |
| Gel strength at 10 min., | 67 | 20 | 47 | 31 |
| lb/100 ft² | | | | |
| Electrical stability, V* | 612 | 647 | 738 | 923 |
| Plastic viscosity, cP | 86 | 33 | 64 | 32 |
| Yield Point, lb/100 ft² | 54 | 11 | 57 | 43 |

*Reported values are an average of two runs.

TABLE 7K
168.75 mL tribromocumene
56.25 mL synthetic oil fluid
Hot-rolling temperature: 177 °C

| | Run | | | |
|---|---|---|---|---|
| | 1 | 2 | 3 | 4 |
| | Before hot-rolling | | After hot-rolling | |
| | Temperature | | | |
| | 78 °F | 148 °F | 74 °F | 153 °F |
| | (25.6 °C) | (64.4 °C) | (23.3 °C) | (67.2 °C) |
| | Shear | Shear | Shear | Shear |
| Stirrer speed, RPM | stress | stress | stress | stress |
| 600 | 209 | 80 | 223 | 104 |
| 300 | 122 | 47 | 146 | 72 |
| 200 | 98 | 33 | 114 | 57 |
| 100 | 64 | 21 | 78 | 39 |
| 6 | 30 | 10 | 31 | 21 |
| 3 | 28 | 9 | 28 | 19 |
| Gel strength at 10 sec., | 50 | 18 | 34 | 25 |
| lb/100 ft² | | | | |
| Gel strength at 10 min., | 62 | 20 | 48 | 29 |
| lb/100 ft² | | | | |
| Electrical stability, V* | 558 | 615 | 708 | 942 |
| Plastic viscosity, cP | 87 | 33 | 77 | 32 |
| Yield Point, lb/100 ft² | 35 | 14 | 69 | 40 |

*Reported values are an average of two runs.

TABLE 7L
168.75 mL tribromocumene
56.25 mL synthetic oil fluid
Hot-rolling temperature: 177 °C

| | Run | | | |
|---|---|---|---|---|
| | 1 | 2 | 3 | 4 |
| | Before hot-rolling | | After hot-rolling | |
| | Temperature | | | |
| | 80 °F | 150 °F | 74 °F | 153 °F |
| | (26.7 °C) | (65.6 °C) | (23.3 °C) | (67.2 °C) |
| | Shear | Shear | Shear | Shear |
| Stirrer speed, RPM | stress | stress | stress | stress |
| 600 | 190 | 68 | 184 | 90 |
| 300 | 117 | 39 | 117 | 61 |
| 200 | 87 | 28 | 94 | 47 |
| 100 | 58 | 18 | 63 | 33 |
| 6 | 28 | 10 | 24 | 17 |
| 3 | 26 | 9 | 21 | 16 |
| Gel strength at 10 sec., | 48 | 15 | 27 | 22 |
| lb/100 ft² | | | | |
| Gel strength at 10 min., | 64 | 18 | 42 | 26 |
| lb/100 ft² | | | | |
| Electrical stability, V* | 583 | 665 | 724 | 890 |
| Plastic viscosity, cP | 73 | 29 | 67 | 29 |
| Yield Point, lb/100 ft² | 44 | 10 | 50 | 32 |

*Reported values are an average of two runs.

TABLE 7M
112.5 mL tribromocumene
112.5 mL synthetic oil fluid
Fluid loss filter cake thickness: 1/16-inch (0.159 cm)

| Sq. root | | | Incremental | Cumulative | Fluid loss |
|---|---|---|---|---|---|
| of time | Time | | loss | loss | (API volume) |
| 1 | 1 | min. | 0 mL | 0 mL | 0 mL |
| 2.236068 | 5 | min. | 0 mL | 0 mL | 0 mL |
| 2.738613 | 7.5 | min. | 0 mL | 0 mL | 0 mL |
| 3.16227766 | 10 | min. | 0 mL | 0 mL | 0 mL |
| 3.872983 | 15 | min. | 0 mL | 0 mL | 0 mL |
| 4.472136 | 20 | min. | 0 mL | 0 mL | 0 mL |
| 5 | 25 | min. | 0 mL | 0 mL | 0 mL |
| 5.477226 | 30 | min. | 0 mL | 0 mL | 0 mL |

TABLE 7N
168.75 mL tribromocumene
56.25 mL synthetic oil fluid
Fluid loss filter cake thickness: ¼-inch (0.635 cm)

| Sq. root | | | Incremental | | Cumulative | | Fluid loss | |
|---|---|---|---|---|---|---|---|---|
| of time | Time | | loss | | loss | | (API volume) | |
| 1 | 1 | min. | 0 | mL | 0 | mL | 0 | mL |
| 2.236068 | 5 | min. | 0 | mL | 0 | mL | 0 | mL |
| 2.738613 | 7.5 | min. | 0.3 | mL | 0.3 | mL | 0.6 | mL |
| 3.16227766 | 10 | min. | 0.1 | mL | 0.4 | mL | 0.8 | mL |
| 3.872983 | 15 | min. | 0.3 | mL | 0.7 | mL | 1.4 | mL |
| 4.472136 | 20 | min. | 0.2 | mL | 0.9 | mL | 1.8 | mL |
| 5 | 25 | min. | 0.2 | mL | 1.1 | mL | 2.2 | mL |
| 5.477226 | 30 | min. | 0.2 | mL | 1.3 | mL | 2.6 | mL |

TABLE 7O
168.75 mL tribromocumene
56.25 mL synthetic oil fluid
Fluid loss filter cake thickness: ¼-inch (0.635 cm)

| Sq. root | | | Incremental | | Cumulative | | Fluid loss | |
|---|---|---|---|---|---|---|---|---|
| of time | Time | | loss | | loss | | (API volume) | |
| 1 | 1 | min. | 0.8 | mL | 0.8 | mL | 1.6 | mL |
| 2.236068 | 5 | min. | 0.6 | mL | 1.4 | mL | 2.8 | mL |
| 2.738613 | 7.5 | min. | 0.1 | mL | 1.5 | mL | 3 | mL |
| 3.16227766 | 10 | min. | 0.1 | mL | 1.6 | mL | 3.2 | mL |
| 3.872983 | 15 | min. | 0.4 | mL | 2.0 | mL | 4 | mL |
| 4.472136 | 20 | min. | 0.4 | mL | 2.4 | mL | 4.8 | mL |
| 5 | 25 | min. | 0.2 | mL | 2.6 | mL | 5.2 | mL |
| 5.477226 | 30 | min. | 0.3 | mL | 2.9 | mL | 5.8 | mL |

TABLE 7P
168.75 mL tribromocumene
56.25 mL synthetic oil fluid
Fluid loss filter cake thickness: ¼-inch (0.635 cm)

| Sq. root | | | Incremental | | Cumulative | | Fluid loss | |
|---|---|---|---|---|---|---|---|---|
| of time | Time | | loss | | loss | | (API volume) | |
| 1 | 1 | min. | 3.9 | mL | 3.9 | mL | 7.8 | mL |
| 2.236068 | 5 | min. | 5 | mL | 8.9 | mL | 17.8 | mL |
| 2.738613 | 7.5 | min. | 1.9 | mL | 10.8 | mL | 21.6 | mL |
| 3.16227766 | 10 | min. | 1.9 | mL | 12.7 | mL | 25.4 | mL |
| 3.872983 | 15 | min. | 2.1 | mL | 14.8 | mL | 29.6 | mL |
| 4.472136 | 20 | min. | 2.3 | mL | 17.1 | mL | 34.2 | mL |
| 5 | 25 | min. | 2.5 | mL | 19.6 | mL | 39.2 | mL |
| 5.477226 | 30 | min. | 2.4 | mL | 22 | mL | 44 | mL |

Further embodiments of the invention include, without limitation:
A) A liquid weighting agent containing two or more bromine atoms and having a bromine content of about 50 wt % or more, which liquid weighting agent is selected from
   a) a bromine-containing terminal alkene having three or more carbon atoms in the molecule
   b) a bromine-containing internal alkene having four or more carbon atoms in the molecule;
   c) a tribromocumene mixture comprising 2,4,5-tribromocumene and one or more unsubstituted tribromobenzenes, wherein 2,4,5-tribromocumene is about 70 wt % or more relative to the total weight of the tribromocumene mixture, and the unsubstituted tribromobenzenes are about 5 wt % or more, relative to the total weight of the mixture; and
   d) any two or more of the foregoing liquid weighting agents.
B) A liquid weighting agent as in A) wherein the bromine-containing terminal alkene has three to about six carbon atoms in the molecule and/or has two or more bromine atoms bound to alkenyl carbon atoms.
C) A liquid weighting agent as in A) wherein the bromine-containing internal alkene has four to about eight carbon atoms in the molecule and/or has two or more bromine atoms bound to alkenyl carbon atoms.
D) A liquid weighting agent as in A) wherein the tribromocumene mixture has 2,4,5-tribromocumene in an amount of about 70 wt % to about 75 wt %, relative to the total weight of the tribromocumene mixture, and/or unsubstituted tribromobenzenes in an amount of 5 wt % to about 10 wt %, relative to the total weight of the tribromocumene mixture.
E) A liquid weighting agent as in A) wherein the tribromocumene mixture contains as unsubstituted tribromobenzenes, 1,2,4-tribromobenzene in an amount of about 5 wt % or more, relative to the total weight of the tribromocumene mixture, and optionally 1,3,5-tribromobenzene, in an amount of about 0.75 wt % or more, relative to the total weight of the tribromocumene mixture.
F) A liquid weighting agent as in E) wherein the tribromocumene mixture contains 1,2,4-tribromobenzene in an amount of about 5 wt % to about 10 wt %, relative to the total weight of the tribromocumene mixture, and optionally 1,3,5-tribromobenzene, in an amount of about 0.75 wt % to about 1.5 wt %, relative to the total weight of the tribromocumene mixture.
G) A liquid weighting agent as in E) or F) wherein other brominated cumene species are present in the tribromocumene mixture, and the total amount of other brominated cumene species is about 5 wt % to about 15 wt %, relative to the total weight of the tribromocumene mixture
H) A liquid weighting agent as in any of E)-G) wherein 2,3,5-tribromocumene, 3,4,5-tribromocumene, and/or 2,3,4,5-tetrabromocumene are present in the tribromocumene mixture, in a total amount of about 5 wt % or more, relative to the total weight of the relative to the total weight of the tribromocumene mixture.
I/K) A liquid weighting agent as in any of A)-H) wherein the liquid weighting agent is 1,2-dibromo-1-hexene; 2,3-dibromo-2-butene; 2,3,4,5-tetrabromo-2,4-hexadiene; and/or a tribromocumene mixture having 2,4,5-tribromocumene in an amount of about 70 to about 73 wt %, 2,3,5-tribromocumene in an amount of about 3 wt % to about 5 wt %, 3,4,5-tribromocumene in an amount of about 1 wt % to about 2.5 wt %, 2,3,4,5-tetrabromocumene in an amount of about 7.5 wt % to about 9.5 wt %, 1,2,4,5-tetrabromobenzene in an amount of about 0.5 wt % to about 1.5 wt %, 1,2,4-tribromobenzene in an amount of about 6 wt % to about 8 wt %, and 1,3,5-tribromobenzene in an amount of about 1.25 wt % to about 2.5 wt %, all relative to the total weight of the tribromocumene mixture.
J/L) A liquid weighting agent as in any of A)-I) wherein one or more additives selected from unsaturated diesters that do not contain bromine and/or saturated triesters that do not contain bromine are included with the liquid weighting agent, wherein the unsaturated diester has at least eight carbon atoms and is represented by the formula

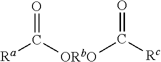

where R^{a} and R^{c} are hydrocarbyl groups, and R^{b} is an alkene-containing moiety, and wherein the saturated triester has at least seven carbon atoms and is represented by the formula

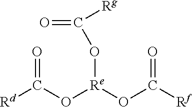

where R^{d}, R^{e}, R^{f}, and R^{g} are hydrocarbyl groups.
K) A liquid weighting agent as in J) wherein the unsaturated diester is but-2-ene-1,4-diyl bis(butyrate), and wherein the saturated triester is propane-1,2,3-triyl tributyrate.
L) A liquid weighting agent as in any of A)-K) which is
   in a mixture with an oil, wherein there is about 0.1 to about 99.9 wt % liquid weighting agent in the mixture with the oil; or
   in combination with a hydraulic fluid.
M) An oil-based well fluid comprising a liquid weighting agent of any of A)-k).
N) An oil-based well fluid as in M) wherein the well fluid is a drilling fluid, a completion fluid, a drill-in fluid, a tractor fluid, a camera fluid, or a workover fluid.
O) A method of treating a wellbore, said method comprising introducing into the wellbore a fluid comprising a liquid weighting agent as in any of A)-K).
P) A method as in O) wherein the fluid is a drilling fluid, a completion fluid, a drill-in fluid, a tractor fluid, a camera fluid, or a workover fluid.
Q) A method for minimizing or relieving a sustained casing pressure in an annulus of a wellbore, wherein the annulus contains a first fluid having a density, the method comprising introducing a second fluid into the annulus, wherein the second fluid has a density greater than the density of the first fluid and the second fluid is immiscible with the first fluid, characterized in that the second fluid comprises a liquid weighting agent of any of A)-K).
R) A process for preparing a tribromocumene mixture comprising 2,4,5-tribromocumene and one or more unsubstituted tribromobenzenes, which process comprises contacting, under an inert atmosphere in a liquid organic medium, cumene and a brominating agent in the presence of a catalytic amount of aluminum chloride.
S) A process as in R) wherein the brominating agent is elemental bromine; and/or aluminum chloride is in an amount of about 0.2 wt % to about 0.6 wt % relative to the cumene.
T) A process as in S) wherein the aluminum chloride is in an amount of about 0.3 wt % to about 0.5 wt % relative to the cumene.

Components referred to by chemical name or formula anywhere in the specification or claims hereof, whether referred to in the singular or plural, are identified as they exist prior to coming into contact with another substance referred to by chemical name or chemical type (e.g., another component, a solvent, or etc.). It matters not what chemical changes, transformations and/or reactions, if any, take place in the resulting mixture or solution as such changes, transformations, and/or reactions are the natural result of bringing the specified components together under the conditions called for pursuant to this disclosure. Thus the components are identified as ingredients to be brought together in connection with performing a desired operation or in forming a desired composition. Also, even though the claims hereinafter may refer to substances, components and/or ingredients in the present tense (“comprises”, “is”, etc.), the reference is to the substance, component or ingredient as it existed at the time just before it was first contacted, blended or mixed with one or more other substances, components and/or ingredients in accordance with the present disclosure. The fact that a substance, component or ingredient may have lost its original identity through a chemical reaction or transformation during the course of contacting, blending or mixing operations, if conducted in accordance with this disclosure and with ordinary skill of a chemist, is thus of no practical concern.

The invention may comprise, consist, or consist essentially of the materials and/or procedures recited herein.

As used herein, the term “about” modifying the quantity of an ingredient in the compositions of the invention or employed in the methods of the invention refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term about also encompasses amounts that differ due to different equilibrium conditions for a composition resulting from a particular initial mixture. Whether or not modified by the term “about”, the claims include equivalents to the quantities.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

This invention is susceptible to considerable variation in its practice. Therefore the foregoing description is not intended to limit, and should not be construed as limiting, the invention to the particular exemplifications presented hereinabove.

1. A liquid weighting agent containing two or more bromine atoms and having a bromine content of about 50 wt % or more, which liquid weighting agent is selected from
a) a bromine-containing terminal alkene having three or more carbon atoms in the molecule;
b) a bromine-containing internal alkene having four or more carbon atoms in the molecule;
c) a tribromocumene mixture comprising 2,4,5-tribromocumene and one or more unsubstituted tribromobenzenes, wherein 2,4,5-tribromocumene is about 70 wt % or more relative to the total weight of the tribromocumene mixture, and the unsubstituted tribromobenzenes are about 5 wt % or more, relative to the total weight of the mixture; and
d) any two or more of the foregoing liquid weighting agents.

2. A liquid weighting agent as in claim 1 wherein
the bromine-containing terminal alkene has three to about six carbon atoms in the molecule;
the bromine-containing internal alkene has four to about eight carbon atoms in the molecule;
the tribromocumene mixture has 2,4,5-tribromocumene in an amount of about 70 wt % to about 75 wt %, relative to the total weight of the tribromocumene mixture, and/or unsubstituted tribromobenzenes in an amount of 5 wt % to about 10 wt %, relative to the total weight of the tribromocumene mixture.

3. A liquid weighting agent as in claim 1 wherein
the bromine-containing terminal alkene has two or more bromine atoms bound to alkenyl carbon atoms;
the bromine-containing internal alkene has two or more bromine atoms bound to alkenyl carbon atoms;
the tribromocumene mixture contains as unsubstituted tribromobenzenes, 1,2,4-tribromobenzene in an amount of about 5 wt % or more, relative to the total weight of the tribromocumene mixture, and optionally 1,3,5-tribromobenzene, in an amount of about 0.75 wt % or more, relative to the total weight of the tribromocumene mixture.

4. A liquid weighting agent as in claim 3 wherein the tribromocumene mixture contains 1,2,4-tribromobenzene in an amount of about 5 wt % to about 10 wt %, relative to the total weight of the tribromocumene mixture, and optionally 1,3,5-tribromobenzene, in an amount of about 0.75 wt % to about 1.5 wt %, relative to the total weight of the tribromocumene mixture.

5. A liquid weighting agent as in claim 3 wherein the tribromocumene mixture contains brominated cumene species other than 2,4,5-tribromocumene, and the total amount of other brominated cumene species is about 5 wt % to about 15 wt %, relative to the total weight of the tribromocumene mixture.

6. A liquid weighting agent as in claim 3 wherein 2,3,5-tribromocumene, 3,4,5-tribromocumene, and/or 2,3,4,5-tetrabromocumene are present in the tribromocumene mixture, in a total amount of about 5 wt % or more, relative to the total weight of the relative to the total weight of the tribromocumene mixture.

7. A liquid weighting agent as in claim 1 wherein the liquid weighting agent is 1,2-dibromo-1-hexene; 2,3-dibromo-2-butene; 2,3,4,5-tetrabromo-2,4-hexadiene; and/or a tribromocumene mixture having 2,4,5-tribromocumene in an amount of about 70 to about 73 wt %, 2,3,5-tribromocumene in an amount of about 3 wt % to about 5 wt %, 3,4,5-tribromocumene in an amount of about 1 wt % to about 2.5 wt %, 2,3,4,5-tetrabromocumene in an amount of about 7.5 wt % to about 9.5 wt %, 1,2,4,5-tetrabromobenzene in an amount of about 0.5 wt % to about 1.5 wt %, 1,2,4-tribromobenzene in an amount of about 6 wt % to about 8 wt %, and 1,3,5-tribromobenzene in an amount of about 1.25 wt % to about 2.5 wt %, all relative to the total weight of the tribromocumene mixture.

8. A liquid weighting agent as in claim 1 wherein one or more additives selected from unsaturated diesters that do not contain bromine and/or saturated triesters that do not contain bromine are included with the liquid weighting agent, wherein the unsaturated diester has at least eight carbon atoms and is represented by the formula 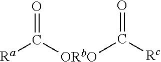
where R^{a} and R^{c} are hydrocarbyl groups, and R^{b} is an alkene-containing moiety, and wherein the saturated triester has at least seven carbon atoms and is represented by the formula 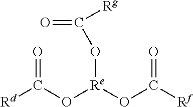
where R^{d}, R^{e}, R^{f}, and R^{g} are hydrocarbyl groups.

9. A liquid weighting agent as in claim 8 wherein the unsaturated diester is but-2-ene-1,4-diyl bis(butyrate), and wherein the saturated triester is propane-1,2,3-triyl tributyrate.

10. A liquid weighting agent as in claim 1 in a mixture with an oil, wherein there is about 0.1 to about 99.9 wt % liquid weighting agent in the mixture with the oil.

11. A liquid weighting agent as in claim 1 in combination with a hydraulic fluid.

12. An oil-based well fluid comprising a liquid weighting agent of claim 1.

13. An oil-based well fluid as in claim 12 wherein the well fluid is a drilling fluid, a completion fluid, a drill-in fluid, a tractor fluid, a camera fluid, or a workover fluid.

14. A method of treating a wellbore, said method comprising introducing into the wellbore a fluid comprising a liquid weighting agent as in claim 1.

15. A method as in claim 14 wherein the fluid is a drilling fluid, a completion fluid, a drill-in fluid, a tractor fluid, a camera fluid, or a workover fluid.

16. A method for minimizing or relieving a sustained casing pressure in an annulus of a wellbore, wherein the annulus contains a first fluid having a density, the method comprising introducing a second fluid into the annulus, wherein the second fluid has a density greater than the density of the first fluid and the second fluid is immiscible with the first fluid, characterized in that the second fluid comprises a liquid weighting agent of claim 1.

17. Each of the following separately, as a new composition of matter:
2,3,4,5-tetrabromo-2,4-hexadiene;
a tribromocumene mixture comprising 2,4,5-tribromocumene and one or more unsubstituted tribromobenzenes, wherein 2,4,5-tribromocumene is about 70 wt % or more relative to the total weight of the tribromocumene mixture, and the unsubstituted tribromobenzenes are about 5 wt % or more, relative to the total weight of the mixture.

18. A process for preparing a tribromocumene mixture comprising 2,4,5-tribromocumene and one or more unsubstituted tribromobenzenes, which process comprises contacting, under an inert atmosphere in a liquid organic medium, cumene and a brominating agent in the presence of a catalytic amount of aluminum chloride.

19. A process as in claim 18 wherein
the brominating agent is elemental bromine; and/or
aluminum chloride is in an amount of about 0.2 wt % to about 0.6 wt % relative to the cumene.
